(19) United States
(12) Patent Application Publication
(10) Pub. No.: US 2018/0168839 A1
(43) Pub. Date: Jun. 21, 2018

(54) METHODS AND DEVICES FOR CONFIRMING PLACEMENT OF A DEVICE WITHIN A CAVITY

(71) Applicant: Allurion Technologies, Inc, Natick, MA (US)

(72) Inventors: Matthew J. LAPINSKI, Andover, MA (US); Matthew S. LAKE, Millis, MA (US)

(73) Assignee: Allurion Technologies, Inc, Natick, MA (US)

(21) Appl. No.: 15/867,508

(22) Filed: Jan. 10, 2018

Related U.S. Application Data
(63) Continuation of application No. PCT/US2016/041742, filed on Jul. 11, 2016.
(60) Provisional application No. 62/562,882, filed on Sep. 25, 2017.
(60) Provisional application No. 62/191,264, filed on Jul. 10, 2015.

(51) Int. Cl. A61F 5/00; A61B 5/06

(52) U.S. Cl. CPC A61F 5/0036; A61F 5/0026; A61F 5/0089; A61B 5/06

(57) ABSTRACT

Methods and devices for confirming the location of an ingested device within a cavity.

### RELATED APPLICATIONS

This application is a continuation of PCT Application No. PCT/US2016/041742 filed Jul. 11, 2016, which is a non-provisional of U.S. Provisional Application No. 62/191,264 filed Jul. 10, 2015. This application is also a non-provisional of U.S. Provisional Application 62/562,282 filed Sep. 25, 2017, the entirety of each of which is incorporated by reference in its entirety.

### FIELD OF THE INVENTION

The present invention generally relates to the field of gastric devices and more particularly to the field of ingestible gastric devices. In particular, the present invention relates to methods and apparatuses for confirming the location of an ingested gastric device along and within the gastro-intestinal tract.

### BACKGROUND

In medical applications where a device is positioned within the body in a non-invasive or minimally invasive manner it is important to confirm placement of the device in the desired location prior to deployment or actuation of the device. This is especially true for gastric applications where the gastric device can assist overweight and obese patients for whom surgical obesity procedures are not appropriate, not efficacious or unaffordable interventions.

Current gastric devices are intended to provide an effective treatment for obesity and can even be useful for a wider patient population when applied to clinical areas outside of obesity.

When the device is swallowed, or otherwise positioned it is important that the device is properly positioned within the stomach prior to inflation or deployment from any delivery structure coupled to the device. There is a window of time to activate the device to avoid complications. At the start, the device must sufficiently traverse through the esophagus and advance clear of the esophageal sphincter. However, the device must be actuated prior to passing into the pyloric sphincter and into the small intestines.

The residence time of items ingested into the stomach is highly variable between patients. The timing of activation, inflation, and/or disengagement of the device is important. The timing must match the window of time that prevents premature inflation in the esophagus or delayed inflation in the intestines. Missing the window can result in blockage or damage of either the esophagus, intestines, esophageal sphincter, or pyloric sphincter.

There is a trend to verify positioning within the stomach using non-invasive imaging such as radiography. After a patient swallows a device, radiography can provide visualization of the device or other structure. Such radiographic imaging includes x-ray or fluoroscopy techniques that provide real-time images of the balloon using radiation. However, radiation involves effects that can be harmful to the body of the patient and/or medical caregiver if such exposure is prolonged or administered in high doses. Fluoroscopy typically uses lower doses of radiation but when repeated use may create a risk of harm to a patient and/or medical caregiver. Further, there is the risk of accidental administration of too high of a dose to a patient. Also, administration of devices might be limited to those physicians and/or locations having radiography equipment.

Ultrasound-based systems and methods can be an alternative to radiography to detect objects and measure distances. Medical sonography (ultrasonography) is an ultrasound-based diagnostic medical imaging technique used to visualize body structures and devices real time images. However, the use of ultrasound still introduces added cost and time to the procedure and can limit administration of the device to those environments where ultrasound equipment is available.

There remains a need to confirm placement of a device, such as a gastric device, while addressing the problems discussed above.

### BRIEF SUMMARY OF THE INVENTION

The present invention provides, in one variation, a motion sensor attached in close proximity to a gastric device, the device intended to be deployed in the gastro-intestinal (GI) tract of an animal, generally a human patient. The device is attached to the end of a conduit, filament, cord, or other extended member, the non-device end of which is retained outside the body. In one aspect, after the device is deployed (typically by swallowing), the patient is moved or rocked gently, or instructed to move or rock. This motion is sensed by the motion sensor. The range and characteristics of the sensed motion are indicative of the location of the motion sensor along the GI tract. Specifically, the device will swing more freely when it is in a relatively open space like the stomach than when it is in a relatively confined space like the esophagus.

The present disclosures include methods and devices for determining placement of a therapeutic and/or monitoring device within a cavity of a body of a patient. In one example, such a method can include inserting an extension member having a device coupled to a distal portion of the extension member into a body of a patient; advancing the extension member and the gastric device through a lumen in the body of the patient; receiving a first plurality data comprising a motion of the gastric device over a period of time; and comparing the plurality of data to determine the motion of the device over at least a first sub-period of time against the motion of the device over at least a second sub-period of time and confirming a location of the device in the cavity by identifying whether the first sub-period of time or the second sub-period of time comprises a greater degree of motion.

As noted herein, one of the aspects of the methods and devices is that when the therapeutic device is attached to an extension member, including but not limited to a conduit, catheter, signal transport, tubing or similar extension member, the therapeutic device can be induced into a pendulum type of motion given that the extension member is located within a passageway while the therapeutic device is located in a cavity (e.g., the esophagus and stomach). It is noted that the device and/or methods can include any number of extension members, such as a conduit and a signal transport.

A variation of the method includes detaching the device from the extension member upon confirming the location of the device in the cavity. The methods and systems described herein can be used for confirming the position of any device within a cavity of the body, including but not limited to a stomach. Such devices can be therapeutic, diagnostic, monitoring, and/or drug dispensing. In one variation, the devices can comprise a gastric device, including but not limited to an inflatable and/or expandable gastric device.

Variations of the method include receiving the first plurality of data from a first motion sensor configured to produce the plurality of data. The first motion sensor can be coupled to the device or can be coupled to the extension member. In variations, a plurality of sensor members are used and positioned along the extension member and/or device.

The method can further include receiving a second plurality of data comprising a motion of the extension member over a period of time; and further comparing the second plurality of data against the first plurality of data to confirm that the device is located within the cavity upon determining that the motion of the extension member is less than the motion of the device.

In additional variations, the method can further comprise a signal transport member coupled to the first motion sensor, where the signal transport member conveys the plurality of data. The signal transport member can also be coupled to the second motion sensor.

Another variation of the method includes securing a proximal portion of the extension member while advancing the extension member and device through the lumen in the body of the patient.

In order to assist in detection of movement of the device, the method can also include inducing motion of the device within the patient. Inducing motion can comprise causing the patient to move or causing the device and/or extension member to move in the pendulum motion. For example, the patient can physically move or can be positioned on a structure (such as a bed, chair, or other platform or mechanized structure) that causes movement. Alternatively, or in combination, inducing motion of the device within the patient comprises applying a force to the device through the extension member.

The method can also include displaying the plurality of signals over the period of time. Moreover, the method can further include comparing a first sub-period of the plurality of signals against a second sub-period of the plurality of signals and identifying a location of the device within the cavity by confirming that a degree of motion of the second-sub period is greater than a degree of motion of the first sub-period.

The present disclosure also includes medical systems for confirming a location of a device. For example, such a medical system can include an extension member having a proximal portion and a distal portion; a device coupled to the distal portion of the extension member; a first motion sensor located adjacent to the distal portion of the extension member, the motion sensor configured to generate a plurality of signals representative of a movement of the first motion sensor; and a signal processing unit configured to receive the plurality of signals over a period of time.

The medical system can include variations having at least a second motion sensor coupled to a portion of the extension member located between the proximal portion and the first motion sensor, where the second motion sensor is configured to generate a second plurality of signals regarding a movement of the second motion sensor over the period of time.

In an additional variation, the medical system further comprises a plurality of conductive members extending through the extension member, where the plurality of conductive members electrically couples the signal processing unit to the first sensor.

Alternatively, or in combination, the medical system can include a wireless transmitting unit configured to transmit the plurality of signals to the signal processing unit.

The medical system can also include a display unit coupled to the signal processing unit, where the display unit is configured to graphically display a range of motion of the first sensor as derived from the plurality of signals, over the period of time.

In an additional variation, the signal processing unit is further configured compare the plurality of signals to determine the motion of the device over at least a first sub-period of time versus the motion of the device over at least a second sub-period of time

Variations of the medical system can also include a signal processing unit that is further configured to confirm a position of the device in a cavity by identifying whether the first sub-period of time or the second sub-period of time comprises a greater degree of motion. The medical system can have a display unit configured to graphically display the ranges of motion of both the first and the second motion sensor over the first and the second sub-periods of time.

The present disclosure also includes a motion detection system for deploying a device in a body cavity. For example the system comprises a first motion sensor, said sensor compatible with an environment of a mammalian gastro-intestinal tract and capable of generating one or more signals related to its position; an electronic signal processor configured to convert the signals produced by the first motion sensor into information related to a position of the sensor; and a signal transport subsystem which conveys the signals produced by the first motion sensor from the sensor to the electronic signal processor, wherein the first motion sensor is disposed in proximity to the gastric device and where the first motion sensor generates a time-series of signals that are a function of the sensor's change of position over time.

In another variation, the present disclosure includes a position determining system for a gastric device comprising: a first motion sensor, said sensor compatible with an environment of a mammalian gastro-intestinal tract and capable of generating one or more signals related to its position; an electronic signal processor configured to convert the signals produced by the first motion sensor into information related to a position of the sensor; and a signal transport subsystem which conveys the signals produced by the first motion sensor from the sensor to the electronic signal processor, wherein the first motion sensor is disposed in proximity to the gastric device and where the first motion sensor generates a time-series of signals that are a function of the sensor's change of position over time.

The positioning determining system can include a signal transport system that comprises one or more extended metallic electrical conductors, said conductors extending from the first motion sensor to the electronic signal processor. Alternatively, or in combination, the signal transport system comprises a wireless communications link configured to communicate between the first motion sensor and the electronic signal processor.

Another method of determining the position of a gastric device within an animal gastro-intestinal tract can comprise attaching, directly or indirectly, a first motion sensor to the gastric device, the first motion sensor being in proximity to the device, the motion sensor having a signal transport system, the signal transport system conveying signals between the motion sensor and an external electronic signal processor; causing the mammal to swallow the gastric device; periodically causing the mammal to rock back and forth across a vertical, upright position; interpreting the output from the electronic signal processor to determine the position of the first motion sensor in the gastro-intestinal tract.

In another variation, a method of determining the position of a gastric device within an animal gastro-intestinal tract can include attaching, directly or indirectly, a first motion sensor to the gastric device or to a filamentary member connected to the gastric device, the first motion sensor being in proximity to the device, the motion sensor having a signal transport system, the signal transport system conveying signals between the motion sensor and an external electronic signal processor; optionally attaching, directly or indirectly, a second motion sensor to the filamentary member connected to the gastric device, the second motion sensor disposed at a pre-determined distance from the gastric device and also having a signal transport system; causing the mammal to swallow the gastric device while retaining the external end of the catheter external to the mammal; periodically causing the mammal to rock back and forth across a vertical, upright position; and interpreting the output from the electronic signal processor to determine the position of the first motion sensor in the gastro-intestinal tract, using the second motion sensor as a reference.

Another variation of the system can include two motions sensors that are attached in proximity to a gastric device. One sensor is disposed in close proximity to the device while the second sensor is disposed at a predetermined distance away from the device and is attached to the extended member. After the device is deployed the first sensor measures the motion of the device and the second sensor measures the motion of the extended member at the predetermined distance away from the device. As described above, the range and characteristics of the sensed motions are indicative of the locations of the motion sensors along the GI tract. Specifically, a sensor will swing more freely when it is in a relatively open space like the stomach than when it is in a relatively confined space like the esophagus. The said second sensor, being deployed further up the extended member than the first sensor, will remain in the esophagus after the first sensor (and the device) enter the stomach. Comparing the sensed motion of the two sensors provides a better indication of when the first sensor has reached the stomach.

In some embodiments, the sensors are connected to an electronics module disposed outside of the body by a multi-wire cable which is routed along the extended member. The electronics module can provide power over the cable to the sensors and is in signal communication with the sensors, transmitting and receiving digital or analog signals as specified by the sensor manufacturer. In other embodiments the sensors operate wirelessly, communicating with the electronics module using short range wireless protocols. The wireless sensors may contain or be mounted in close proximity to power supplies such as batteries or so-called super capacitors.

In another aspect, the sensors are mounted directly or indirectly to the extended member and are removed from the body when the extended member is removed from the body. In other embodiments, there is no extended member needed for the operation of the device, in which case the sensors can be mounted to the multi-wire cable that can function as the extended member.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE SEVERAL VIEWS OF THE DRAWING

The foregoing and other objects, features and advantages of the invention will become apparent from the following description in conjunction with the accompanying drawings, in which reference characters refer to the same parts throughout the different views. The drawings are not necessarily to scale; emphasis has instead been placed upon illustrating the principles of the invention. Of the drawings:

FIG. 1 illustrates a gastric implantable device during deployment;

FIG. 2 is a schematic diagram of the tracking method apparatus;

FIGS. 3A and 3B illustrates the method applied to a single motion sensor apparatus variation;

FIGS. 4A and 4B are notional graphs showing the output of a motion sensor;

FIGS. 5A and 5B illustrates the method applied to a multiple motion sensor variation;

FIG. 6 is a notional graphs showing the output of a motion sensor;

FIG. 7 is the electrical connection diagram of an example three-axis micro accelerometer; and

FIG. 8 is a schematic illustration of printed circuit interface boards for use with the accelerometer of FIG. 7 and a micro-ribbon cable.

FIG. 9 illustrates another variation of methods and devices for tracking a device as described herein.

### DETAILED DESCRIPTION OF THE INVENTION

This following provides examples of methods and devices for confirming placement of a device within a cavity, such as a stomach. Moreover, the methods and devices are not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” “having,” “containing,” “involving,” and variations thereof herein is meant to encompass the items listed thereafter and equivalents thereof as well as additional items. In addition, the embodiments disclosed herein, as well as aspects of each embodiment can be combined as desired.

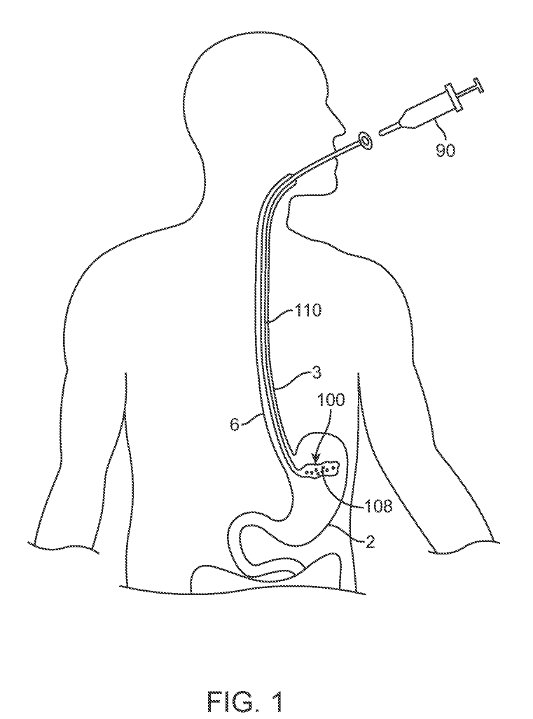

FIG. 1 illustrates a device comprising a gastric balloon 100 deployed to a patient's stomach 2. The balloon 100, in this example, has been swallowed by the patient and has reached stomach 2 by the normal, natural peristalsis process. Balloon 100 is shown in its compact, deployment profile, however it is intended that balloon 100 can expand to a larger, active profile in order to reside in the stomach for a period of time to provide a feeling of fullness to promote weight loss. In alternate variations, the gastric device can be used to deliver substances, devices, or other components for therapeutic and/or medical treatments. The discussion of an expandable balloon is intended for illustrating the aspects of the methods and devices as claimed below.

In the present example the balloon 100 expands to its active profile upon delivery of a filler material 108 into the balloon 100. In the example in FIG. 1 filler material 108 is a liquid that is delivered to balloon 100 through a conduit 110. Filler material 108 may be injected into conduit 110 by a syringe 90.

In other variations, a gastric balloon 100 may be self-expanding, that is, not needing a conduit to carry filler material 108 to balloon 100, or may, in general, be a non-expanding device intended to pass through the gastro-intestinal (GI) tract. In such variations, the gastric balloon or device may be deployed at the end of a length of filamentary material or simply be swallowed.

As discussed above, expanding the balloon 100 before it is positioned within the stomach 2 can lead to undesirable consequences. Similarly, it is generally desirable to know at least the approximate location of any gastric device along the GI tract. One generally accepted method of determining whether the balloon is in the patient's stomach is to perform x-ray or ultrasound imaging, usually in co-operation with radiographic or sonographic tags on the balloon. These position identification/tracking approaches are often not desirable.

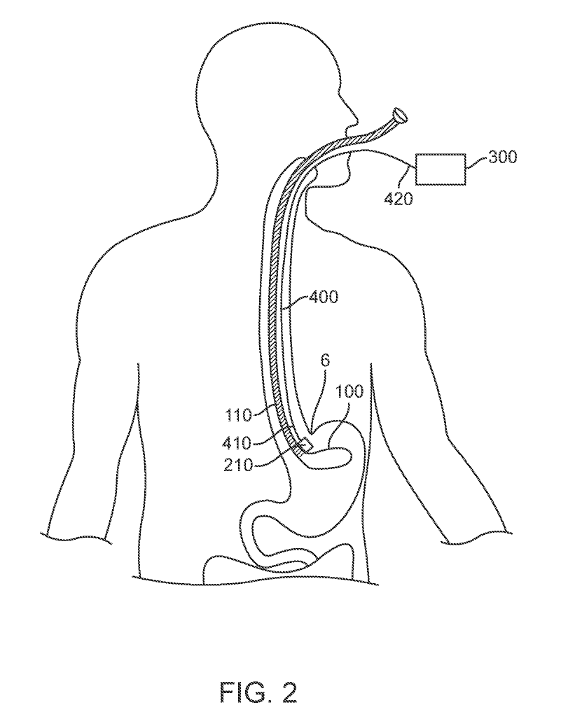

FIG. 2 illustrates an apparatus used in a method of tracking the position of balloon 100 that provides a determination that the balloon has been positioned in the stomach and may be expanded to its active state safely. As shown in FIG. 2 the position tracking apparatus comprises one or more motion sensors 210, an interface and control electronics module 300, and an interconnecting signal transport 400. In one variation, a first motion sensor 210 is attached on device 100 or on signal transport 400 at or near the junction between device 100 and transport 400. A deployed end 410 of interconnecting signal transport 400 is in signal communication with motion sensor 210 and carries the signals generated by motion sensor 210 to control electronics module 300, which is disposed outside the patient's body. Typically transport 400 is attached at least loosely to conduit 110 and is played out or pulled back in concert with conduit 110. An external end 420 of transport 400 is in signal communication with control electronics module 300. Although the electronics module 300 is shown as being coupled via a transport 400, variations include wireless coupling of the electronics module 300 to the sensor 210. For example, the module can be a dedicated unit that receives signals from the sensor 210. Alternatively, or in combination, the module can simply comprise a portable electronic device, such as a smart phone, or other unit, that is configured to receive a wireless signal (e.g., via Bluetooth) from the sensor 210.

In another variation, the position tracking apparatus comprises two substantially identical motion sensors; the above described sensor 210 on or near device 100 and a second motion sensor disposed on signal transport 400, also near deployed end 410 but displaced further away from device 100 than sensor 210 is. For clarity of exposition herein said first sensor 210 may be called the device sensor 210.

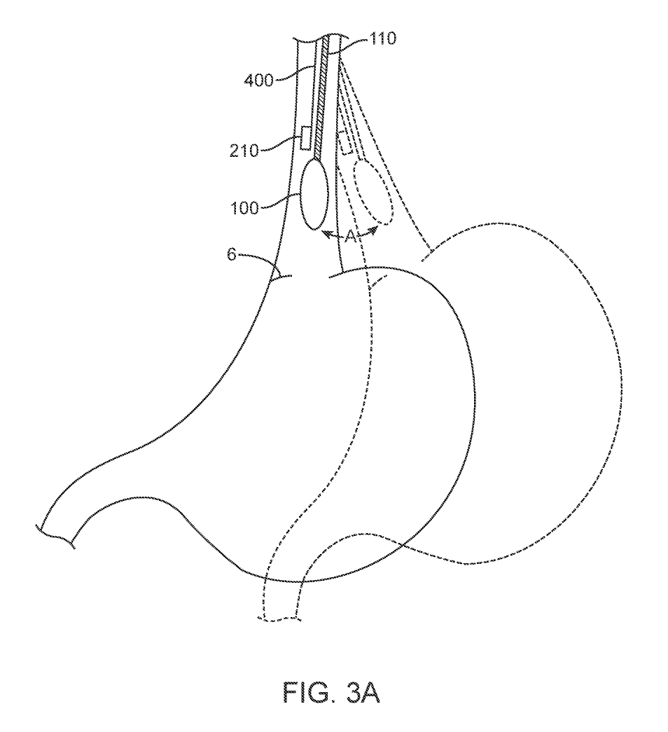

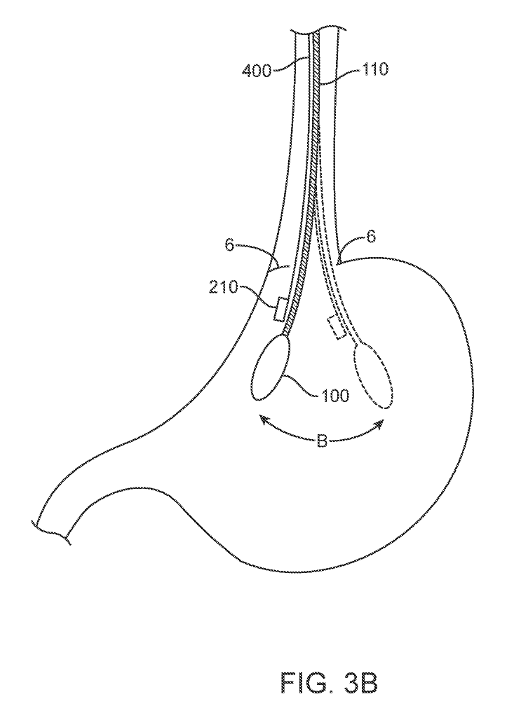

FIG. 3 is an illustration of the method of tracking a device for the variation of the system using just the device sensor 210. FIG. 3A illustrates the position of the balloon when it is close to the esophageal sphincter 6 but has not yet entered stomach 2 while FIG. 3B illustrates the position of the balloon after it has passed esophageal sphincter 6 and entered the stomach. It will be noted that the balloon 100 and conduit 110 are merely representative of any of a number of gastric implantable devices for which the position tracking apparatus and method is applicable. In one variation the apparatus may be attached to any device or object that is intended to be deployed into the gastro-intestinal (GI) tract while attached to an extended string, conduit, catheter, or other filamentary member that is attached at one end to the device or object and has a second end that is retained outside the body and that is long enough to allow the device to reach its intended location along the GI tract while the second end remains outside the body.

The method of tracking the position of device 100 along the GI tract is based on the known differences in free space along the tract. Motion sensors that are located within a confined region of the GI tract, for example the esophagus, will undergo and report motions that are similar to the gross body motions of the patient whereas motion sensors that are located in a less confined region, for example the stomach, will undergo and report a wider range of motion similar to that of a pendulum motion where the device 100 oscillates in movement because of its attachment to the signal transport 400, conduit 110, or any other similar type of extension member. FIGS. 3A and 3B illustrate device 100 with device sensor 210 attached to conduit 110 approximately 3 millimeters away from the junction of the conduit and the device. As will be understood from the following description of the method of tracking, it is generally desirable to locate device sensor 210 as close to the device as is reasonably possible. However, it will also be understood by the designer that the distance between the device sensor and the device can be greater than the minimum possible distance. As long as the device and the sensor are in the same portion of the GI tract, for example in the stomach, the primary effect of increased separation between the sensor and the device is to reduce the sensitivity of the method for indicating the position of the device.

The method of tracking comprises a first step of attaching device sensor 210 and signal transport 400 to conduit 110 or device 100. In some variations device 100 is designed to reside in the GI tract for an extended period of time after being detached from conduit 100, which is withdrawn from the body after detachment. For these resident devices, sensor 210 is typically attached to conduit 110 or to some part of device 100 or its packaging that is not intended to be resident along with device 100 to facilitate withdrawal of the conduit. Alternatively, for these types of devices, sensor 210 is affixed to device 100 and but has a detachable connection to transport 400, allowing transport 400 to be withdrawn with conduit 110.

In other variations where the gastric device does not use any filamentary member, sensor 210 is attached directly to device 100 and device sensor 210 may operate wirelessly, in which case it operates without transport 400 and uses a short-range communications protocol such as Bluetooth to communicate with control electronics module 300. In this wireless variation, sensor 210 must contain or be disposed in proximity to a power source, for example, a battery or so-called super capacitor.

As a second step, device 100 is administered to the patient orally. The swallowed device passes down the esophagus, trailed by conduit 110 and connected transport 400. The administering professional observes the length of conduit/transport that has entered the patient's body. When the length of conduit/transport in the patient's body approximates the estimated length of the patient's esophagus, the administering professional, as a third step, instructs the patient to perform a series of body movements. Typically, these bodily motions may be simple rocking side-to-side motions or leaning forward and backward motions. In other variations the administering professional, as a third step, may stimulate motion of device 100 by other means, for example, by using an actuated bed to rock or roll the patient.

As illustrated in FIG. 3A, when device 100 is in the esophagus the motion detected by motion sensor 210 is substantially identical to the motion of the esophagus, as indicated by double-headed arrow A. However, as illustrated in FIG. 3B, when device 100 is in the stomach by, say, 3 centimeters, the motion detected by motion sensor 210 is substantially greater than the motion of the esophagus due to the pendulum-like behavior of the device, as indicated by double-headed arrow B. The range of motion of the in-stomach device is directly related to the length of conduit 110 by which it hangs from the esophageal sphincter. In addition to having an increased range of motion, device 100, when in the stomach, will also undergo residual oscillations after the patient has stopped moving, again as a result of its pendulum-like suspension whereas a device in the esophagus will stop moving substantially simultaneously with the motion of the esophagus.

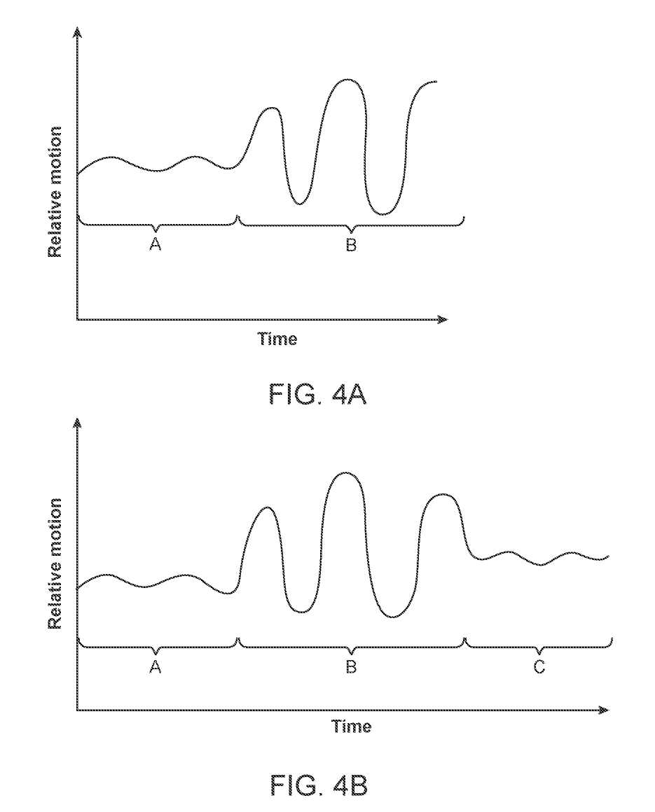

The notional graph in FIG. 4A illustrates the sensed motion (relative to an arbitrary reference position) as a function of time, corresponding to arrows A and B. As is clear from FIG. 4A, the difference in sensed motion between the graph line in the region marked A and the line in the region marked B is a strong indication of whether the sensor is in the esophagus or the stomach. It will also be noted that the signal difference between graph line segment A and graph line segment B is literally an indication of whether the sensor is in a constricted space (segment A) or an open space (segment B), where the constricted space could be, for example, the lower GI tract. Thus, if an orally administered device were allowed to traverse the esophagus, pass through the stomach and enter the intestine, the output signal from the sensor would resemble the notional graph in FIG. 4B, where line segment A represents the signal when the sensor is in the esophagus, segment B represents the signal when the sensor is in the stomach, and segment C, substantially the same as segment A, represents the signal when the sensor is in the small intestine.

Interpretation of the sensor output, which can be considered the last step of the method, can be performed by machine or using human judgement.

It will be understood from the underlying mechanical analysis that the sensitivity of this method for identifying whether device 100 is in the stomach is, to first order, dependent on how far it is disposed into the stomach. That is, when the device is only a few millimeters into the stomach the motion sensor output may not suggest that it has crossed the esophageal sphincter. However, such a false negative is not a problem for applications in which it is important to know positively when the device has reached the stomach. Conversely, the method has a very low, if not zero, false positive rate for indicating that the device is in the stomach—that is, the method does not indicate large-range movement of the device unless the device is in a cavity that permits the pendulum type movement.

This same understanding can be used by the designer to decide the location for device sensor 210. Simply put, the further up the conduit device sensor 210 is put, the less representative its output will be of the device's location and the less sensitive it will be when the device is disposed in the stomach. This desensitization may be useful, for example, for ensuring the device is at a predetermined minimum distance into the stomach. If the device sensor is disposed at that minimum distance away from the device itself, further up the conduit, then the sensor would not even enter the stomach until the device had reached the predetermined minimum distance. Thus, the sensor could not start indicating that it was in the stomach until the device was at the desired minimum distance.

In another variation, the position tracking apparatus comprises two substantially identical motion sensors; the above described sensor 210 on or near device 100 and a second motion sensor 240 disposed on signal transport 400 near deployed end 410 but displaced further away from device 100 than sensor 210 is. That is, second motion sensor 240 is disposed closer to external end 420 than first motion sensor 210. For clarity of exposition said first sensor 210 may be called the device sensor 210 while the second sensor may be called the conduit sensor 240. In this two-sensor variation, signal transport 400 is designed to accommodate the input/output requirements of both sensors. Also, transport 400 further comprises an intermediate connection point 440 at which it is in signal communication with conduit sensor 240.

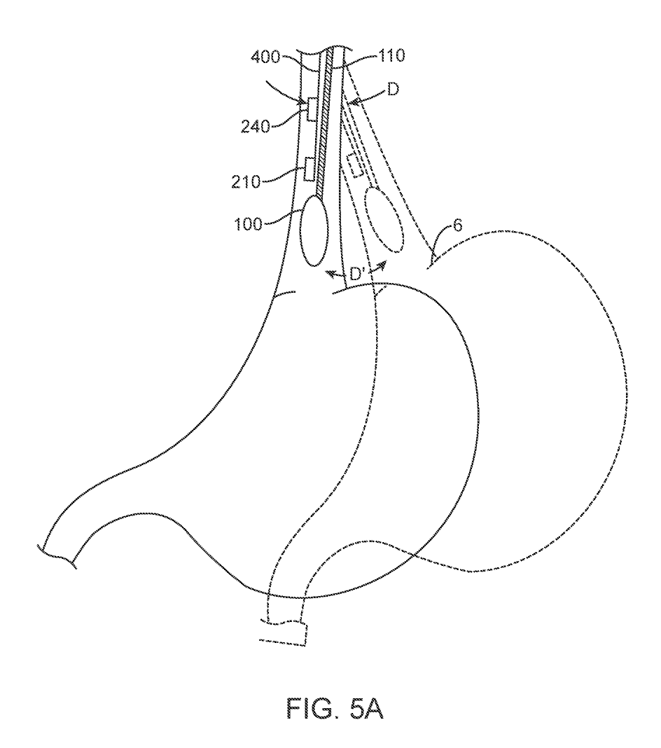

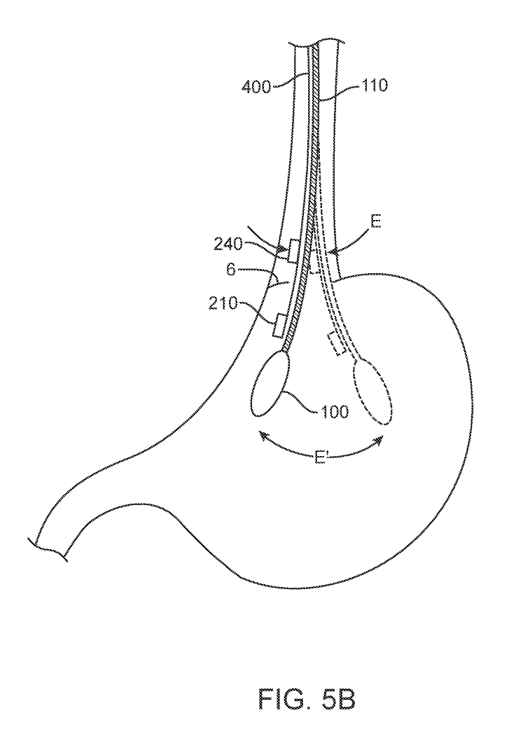

FIGS. 5A and 5B are illustrations of the method of tracking a device for the variation of the system using both device sensor 210 and conduit sensor 240. FIG. 5A illustrates the position of device 100 when it is close to the esophageal sphincter but has not yet entered the stomach while FIG. 5B illustrates the position of device 100 after it has passed the esophageal sphincter and entered the stomach. In both FIG. 5A and FIG. 5B the conduit sensor 240 is disposed in the esophagus. As in FIG. 3, this figure illustrates the motion of the sensors when the patient is instructed to move in, say, a side-to-side rocking motion.

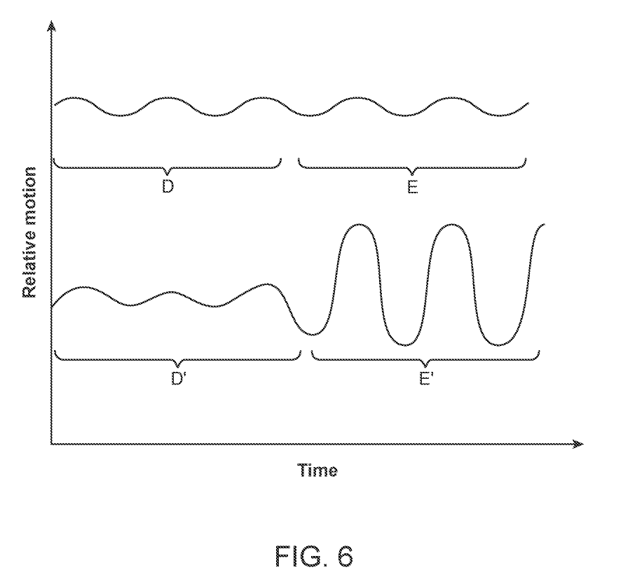

The notional graph in FIG. 6 illustrates the sensed relative (that is, motion about an arbitrary neutral position) as a function of time with line segments marked to correspond to arrows D, D′ and E, E′ in FIG. 5. As is clear from FIG. 6, the difference in sensed motion from the two sensors is small in the region of the graph marked D/D′ and much larger in the region marked E/E′. Comparing the graphs in FIG. 4 and FIG. 6 it is clear to one of skill in the art that the conduit sensor 240 is functioning as a reference for device sensor 210. That is, conduit sensor 240 measures the base (patient's body) motion while device sensor 210 measures the device's motion. Any significant difference between the device's motion and base motion can be ascribed to the device being free of bodily constrain, that is, free from the esophagus. It is this observable difference that indicates the position of device 100 along the gastro-intestinal tract.

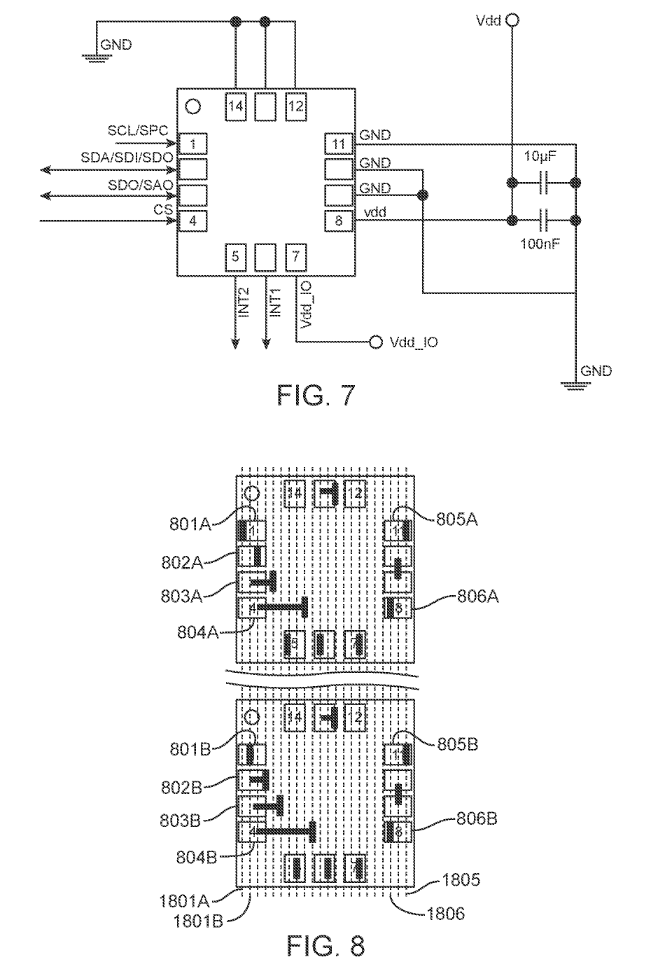

One embodiment of the tracking system comprises small MEMS (Micro Electro-Mechanical Systems) accelerometers as motion sensors. For example, the model LIS2DE MEMS digital output motion sensor, described by the manufacturer, ST Microelectronics of Geneva, Switzerland, as an “ultra-low-power high-performance 3-axis ‘femto’ accelerometer”. This device is packaged in a 2 mm×2 mm×1 mm plastic package and uses about 11 microamps of power at 2.5 volts. This motion sensor may be attached to one end of interconnecting circuit transport 400 via an interface circuit board, where interconnecting circuit transport 400, in one embodiment, comprises a micro ribbon cable such as Temp-Flex MediSpec High-Density Micro-Ribbon Cable, Series No. 100061, available from Molex, 2222 Wellington Court, Lisle, Ill. 60532-1682. This semi-custom ribbon cable is approximately 1 millimeter thick and has a 0.076 millimeter conductor pitch, thereby providing as many as 26 parallel conductors under the footprint of a 2 mm square accelerator. Continuing with the example embodiment, the LIS2DE accelerometer has no more than 11 unique pin connections, as shown in the schematic diagram of FIG. 7, so one, 2-millimeter wide MediSpec cable can easily support both device sensor 210 and conduit sensor 240. In some embodiments, the accelerometers are bonded directly to the cable but, as shown in FIG. 8, more typically a miniature printed wiring board (PWB) provides an interface to better map the accelerometer pins (distributed around the four sides of the square device) to the linear array of conductors in the ribbon cable.

In one variation, as shown in the electrical connection drawing of FIG. 7, the micro-accelerator has 14 electrical connection pins distributed along the four edges of the 2 millimeter square chip. Of the 14 pins, the drawing shows that several may be connected together (for example, pins 12, 13, and 14 are all to be connected to ground), so only 11 actual wire connections are required for each micro-accelerometer. It will be further noted that only 6 (pins 1 through 6) are chip specific. That is, only those 6 pins carry signal/data information and thus represent unique I/O lines for that one chip; the other 5 connections are being made to power or ground sources and are common to all chips.

FIG. 8 schematically illustrates how connection points on the bottom of a pair of PWBs may be arranged to connect the 11 wire connections from each of 2 accelerometer chips to a single, 22 wire, micro-ribbon cable. FIG. 8 is a view looking at the bottoms of the PWBs through a “transparent insulator” micro-ribbon cable. For clarity, the PWB is also “transparent” so the pads on its top surface—the pads that connect to the pins on the bottoms the micro-accelerometers—are visible. The dashed lines in the figure represent the conductors in the ribbon cable and the solid black rectangles are the connection points between the PWB and the cable. It may be assumed that there is a vertical via connecting the cable connecting points to the connection pins directly in line with them. For example, pin 1 on this particular accelerometer is a clock input. Examination of FIG. 8 shows that connection pad 801A is directly above pin 1 of one accelerometer while connection pad 801B is directly above pin 1 of the second accelerometer. However, connection pad 801A is aligned with ribbon cable conductor 1801A while connection pad 801B is aligned with ribbon cable conductor 1801B. Similarly, pad pairs 802A and 802B, 803A and 803B, and 804A and 804B, connecting to pins 2, 3, and 4 on the two accelerometers respectively, are aligned over adjacent pairs of ribbon cable conductors, as are the connection pads for pins 5 and 6. On the other hand, the common power and ground pins—numbers 7 through 14—have connector pins for the two accelerometers aligned with only single cable conductors. For example, ground pins 11 and power pins 8 are immediately below connector pads 805A, 805B and 806A, 806B respectively but the former two pads are both aligned to conductor 1805 and the latter two pads are both aligned to conductor 1806.

As was described above, deployed end 410 of signal transport 400 is disposed at or near device 100 while external end 420 remains external to the patient's body and connects to control electronics module 300. Module 300 is designed to provide power, clock signals, and operational control signals, and to receive interrupt and data signals to and from the accelerometers. Generally, module 300 will serve as an interface between the system and a general purpose digital processor such as a personal computer or tablet. The design and fabrication of module 300 and a software application for the digital processor are easily executed by engineers of ordinary skill in the art, the accelerometers being commercially available devices in commonplace use in electronic devices like cell phones, and will not be discussed in detail herein.

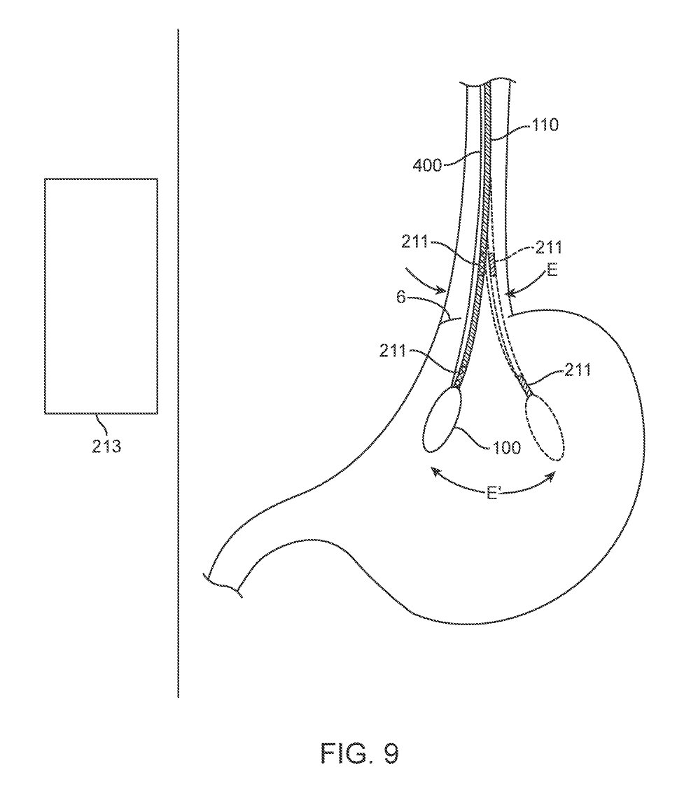

FIG. 9 illustrates another variation of methods and devices for tracking a device as described herein. In this example, the variation of the system uses electromagnetic (EM) coils 211 coupled to a portion of the conduit 110 or device 100. Coil 211 comprises one or more electrical wire loops which, when energized appropriately via transport 400, emits a changing electromagnetic field, which differentiates the field from the substantially unchanging background magnetic fields. Examples of such coils can be found in WO2017127722A1 entitled Low Frequency Electromagnetic Tracking, the entirety of which is incorporated by reference. In this example, at least one coil 211 is used for detection of motion of device 100 (for example when the patient is made to move or rock back and forth). The EM coil 211, in some variations, can be wrapped around an extremely thin core to provide support and, in some instances, to increase the field strength. In some variations, the core has a length on the order of 50 millimeters while in other variations the core is extended the full length of conduit 110. In yet other variations the short core is attached to a stylet that extends the full length of conduit 110. In some variations the core and, if used, stylet is designed to be thin and flexible enough so that its addition does not make the task of swallowing the device 100 more difficult than a device without a tracking feature.

The variation illustrated in FIG. 9 also comprises an external sensor package 213 and, not illustrated, a processing and display station, which may be a personal computer. External sensor package 213 may use multiple spatially dispersed magnetic field sensors to collect signals corresponding to the magnetic fields being emitted from coil 211. The processing station estimates the instantaneous position of coil 211, relative to sensor package 213.

In some variations, the processing station accumulates multiple instantaneous position estimates by which its tracks coil 211 as it progresses down the esophagus and reaches the stomach. In other variations, the processing station can be used to monitor the short term motion of coil 211 looking for the range of motion of the EM coil 211 relative to the sensor package when the patient moves, which is similar to the accelerometer variation discussed above in relation to FIGS. 3A and 3B. As was discussed above, when coil 211 is in the esophagus its range of motion relative to the sensor package is smaller than when the coil is in the open space of the stomach.

In other variations, the EM position sensing system comprises multiple coils 211 disposed along extension member 110, much like accelerometers 210 and 240, which were shown in FIG. 5B. By electrically exciting the multiple coils 211 differently, for example with different frequency sinusoidal currents, the processing station can estimate the multiple coils simultaneously. When multiple coils are disposed along extension member 110 in this fashion, the short-term motion estimates of one coil can be used as a reference for the motion estimates for another coil. This method is similar to the two-accelerometer method discussed previously.

1. A method for determining placement of a device within a cavity of a body of a patient, the method comprising:
inserting an extension member having a device coupled to a distal portion of the extension member into a body of a patient;
advancing the extension member and the gastric device through a lumen in the body of the patient;
receiving a first plurality data comprising a motion of the gastric device over a period of time; and
comparing the plurality of data to determine the motion of the device over at least a first sub-period of time against the motion of the device over at least a second sub-period of time and confirming a location of the device in the cavity by identifying whether the first sub-period of time or the second sub-period of time comprises a greater degree of motion.

2. The method of claim 1, further comprising detaching the device from the extension member upon confirming the location of the device in the cavity.

3. The method of claim 1, further where receiving the first plurality of data comprises receiving the first plurality of data from a first motion sensor configured to produce the plurality of data.

4. The method of claim 3, where the first motion sensor is coupled to the device.

5. The method of claim 3, where the first motion sensor is coupled to the extension member.

6. The method of claim 1, further comprising:
receiving a second plurality of data comprising a motion of the extension member over a period of time; and
further comparing the second plurality of data against the first plurality of data to confirm that the device is located within the cavity upon determining that the motion of the extension member is less than the motion of the device.

7. The method of claim 1, where the extension member comprises a signal transport member coupled to the first motion sensor, where the signal transport member conveys the plurality of data. The method of claim 1, further wherein the signal transport member is coupled to the second motion sensor.

9. The method of claim 1, further comprising securing a proximal portion of the extension member while advancing the extension member and device through the lumen in the body of the patient.

10. The method of claim 1, further comprising inducing motion of the device within the patient.

11. The method of claim 10, where inducing motion of the device within the patient comprises causing the patient to move.

12. The method of claim 10, where inducing motion of the device within the patient comprises applying a force to the device through the extension member.

13. The method of claim 1, further comprising displaying the plurality of signals over the period of time.

14. The method of claim 1, further comprising comparing a first sub-period of the plurality of signals against a second sub-period of the plurality of signals and identifying a location of the device within the cavity by confirming that a degree of motion of the second-sub period is greater than a degree of motion of the first sub-period.

15. A medical system comprising:
an extension member having a proximal portion and a distal portion;
a device coupled to the distal portion of the extension member;
a first motion sensor located adjacent to the distal portion of the extension member, the motion sensor configured to generate a plurality of signals representative of a movement of the first motion sensor; and
a signal processing unit configured to receive the plurality of signals over a period of time.

16. The medical system of claim 15, where the first motion sensor is coupled to the distal portion of the extension member.

17. The medical system of claim 15, where the first motion sensor is coupled to the device.

18. The medical system of claim 15, where the device is removably coupled to the distal portion of the extension member.

19. The medical system of claim 15, further comprising at least a second motion sensor coupled to a portion of the extension member located between the proximal portion and the first motion sensor, where the second motion sensor is configured to generate a second plurality of signals regarding a movement of the second motion sensor over the period of time.

20. The medical system of claim 15, further where the extension member comprises a signal transport member electrically coupled between the signal processing unit and the first motion sensor.

21. The medical system of claim 15, further comprising a plurality of conductive members extending through the extension member, where the plurality of conductive members electrically couples the signal processing unit to the first sensor.

22. The medical system of claim 15, further comprising a wireless transmitting unit configured to transmit the plurality of signals to the signal processing unit.

23. The medical system of claim 15, wherein the first motion sensor is an accelerometer.

24. The medical system of claim 15, further comprising a display unit coupled to the signal processing unit, where the display unit is configured to graphically display a range of motion of the first sensor as derived from the plurality of signals, over the period of time.

25. The medical system of claim 15, where the signal processing unit is further configured to compare the plurality of signals to determine the motion of the device over at least a first sub-period of time versus the motion of the device over at least a second sub-period of time

26. The medical system of claim 25, wherein the signal processing unit is further configured to confirm a position of the device in a cavity by identifying whether the first sub-period of time or the second sub-period of time comprises a greater degree of motion.

27. The medical system of claim 24 where the display unit is configured to graphically display the ranges of motion of both the first and the second motion sensor over the first and the second sub-periods of time.

28. The medical system of claim 19, where motion of second motion sensor provides a reference for the motion of the first motion sensor

29. A method for determining placement of a device within a cavity of a body of a patient, the method comprising:
inserting an extension member having a device coupled to a distal portion of the extension member into a body of a patient;
advancing the extension member and the gastric device through a lumen in the body of the patient with at least one electromagnetic coil coupled thereto;
positioning a sensor device exterior to the body of the patient, where the sensor device is configured determine a position of the at least one electromagnetic coil; and
determining a degree of motion of the device by monitoring movement of the at least one electromagnetic coil to confirm location of the device in the cavity.

30. The method of claim 29, further comprising detaching the device from the extension member upon confirming the location of the device in the cavity.

31. The method of claim 29, where the at least one electromagnetic coil comprises a first electromagnetic coil and a second electromagnetic coil spaced apart on the gastric device and where determining the degree of motion of the device include determining a relative movement between the first electromagnetic coil and the second electromagnetic coil.

32. The method of claim 29, further comprising inducing motion of the device within the patient.

33. The method of claim 32, where inducing motion of the device within the patient comprises causing the patient to move.

34. The method of claim 32, where inducing motion of the device within the patient comprises applying a force to the device through the extension member.
